(19) United States
(12) Patent Application Publication
(10) Pub. No.: US 2015/0209366 A1
(43) Pub. Date: Jul. 30, 2015

(54) COMBINATION OF A MACROCYCLIC PROTEASE INHIBITOR OF HCV, A NON-NUCLEOSIDE HCV INHIBITOR AND RITONAVIR

(71) Applicant: Janssen Pharmaceuticals, Inc., Titusville, NJ (US)

(72) Inventors: Marie-Claude Rouan, Rueil-Malmaison (FR); Jan Snoeys, Westmalle (BE)

(21) Appl. No.: 14/422,005

(22) PCT Filed: Aug. 30, 2013
(86) PCT No.: PCT/IB2013/058138

§ 371 Date: Feb. 17, 2015

(30) Foreign Application Priority Data
Aug. 31, 2012 (EP) 12182551.7
Sep. 25, 2012 (EP) 12185890.6

(51) Int. Cl. A61K 31/55; A61K 31/427; A61K 45/06; A61K 31/4709

(52) U.S. Cl. CPC A61K 31/55; A61K 31/4709; A61K 31/427; A61K 45/06

(57) ABSTRACT

The present invention relates to a combination of a macrocyclic NS3/4A protease inhibitor of HCV, a HCV NS5B polymerase inhibiting non-nucleoside and ritonavir.

### FIELD OF THE INVENTION

The present invention relates to a combination of a macrocyclic NS3/4A protease inhibitor of HCV, a HCV NS5B polymerase inhibiting non-nucleoside and ritonavir.

### BACKGROUND OF THE INVENTION

Hepatitis C virus (HCV), a member of the Flaviviridae family of viruses in the hepacivirus genus, is the leading cause of chronic liver disease worldwide. Although the development of diagnostics and blood screening has considerably reduced the rate of new infections, HCV remains a global health burden due to its chronic nature and its potential for long-term liver damage. There are six major HCV genotypes (1-6) and multiple subtypes (represented by letters). Genotype 1b is predominant in Europe, while genotype 1a is predominant in North America. Genotype is clinically important in determining potential response to therapy and the required duration of such therapy.

HCV is mainly transmitted by blood contact. Following initial acute infection, a majority of infected individuals develops chronic hepatitis because HCV replicates preferentially in hepatocytes but is not directly cytopathic. Over decades, a considerable number of infected persons develop fibrosis, cirrhosis and hepatocellular carcinoma, with chronic HCV infection being the leading cause for liver transplantation. This and the number of patients involved, has made HCV the focus of considerable medical research.

Replication of the genome of HCV is mediated by a number of enzymes, amongst which is HCV NS3/4A serine protease and its associated cofactor, NS4A. Another essential enzyme in this process is NS5B polymerase. Both NS3/4A serine protease and NS5B polymerase are considered to be essential for viral replication and inhibitors of these enzymes are considered drug candidates for HCV treatment.

Various agents have been described that inhibit HCV NS3/4A serine protease. Amongst these, the macrocyclic derivatives are attractive due to their potency and interesting pharmacokinetic profile. WO2007/014926 discloses a series of macrocyclic NS3 serine protease inhibitors. Of these, the compound (1R,4R,6S,15R,17R)-cis-N-[17-[2-(4-isopropylthiazole-2-yl)-7-methoxy-8-methylquinolin-4-yloxy]-13-methyl-2,14-dioxo-3,13-diazatricyclo[13.3.0.0^{4,6}]octadec-7-ene-4-carbonyl]-(cyclopropyl)sulfonamide, which can also be referred to as (1R,4R,6S,7Z,15R,17R)-N-[17-[2-(4-isopropylthiazole-2-yl)-7-methoxy-8-methylquinolin-4-yloxy]-13-methyl-2,14-dioxo-3,13-diazatricyclo[13.3.0.0^{4,6}]octadec-7-ene-4-carbonyl](cyclopropyl)-sulfonamide, i.e. the compound of formula I with the chemical structure depicted hereinafter, is of particular interest.

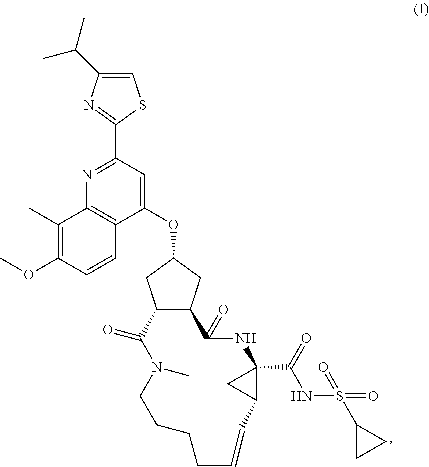 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]

This compound shows pronounced activity against HCV, has an attractive pharmacokinetic profile, and is well-tolerated. This compound can be prepared by the synthesis procedure described in Example 5 of WO2007/014926.

The RNA-dependent RNA polymerase NS5B is essential for replication of the RNA genome. Both nucleoside and non-nucleoside inhibitors of this enzyme are known.

WO2010/003658 describes a number of non-nucleoside inhibitors, one of which is the compound of formula III with the chemical structure depicted hereinafter.

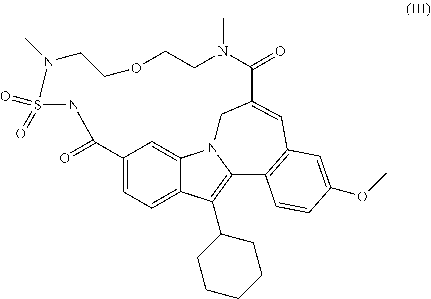 [SMILES: COc1ccc2c(c1)C=C1Cn3c-2c(C2CCCCC2)c2ccc(cc23)C(=O)NS(=O)(=O)N(C)CCOCCN(C)C1=O]

This compound can be prepared by the synthesis procedure described in Example 1 of WO2010/003658.

It may be beneficial to combine two different modes of action in a HCV combination therapy. In addition it would be preferred for such a therapy to have at least one of the following advantages; a lower dose, a lower pill burden, potentially better compliance, reduction of side effects and synergistic effects.

WO97/01349 discloses that a specific compound, ritonavir, may have the ability to inhibit cytochrome P450 monooxygenase activity. It further discloses that the pharmacokinetics of certain HIV protease inhibitors may be improved in rats by combination of said drug with ritonavir (compound of Formula (II)). Adult dosage of ritonavir for boosting HIV protease inhibitors is 100 mg or 200 mg (BID or QD).

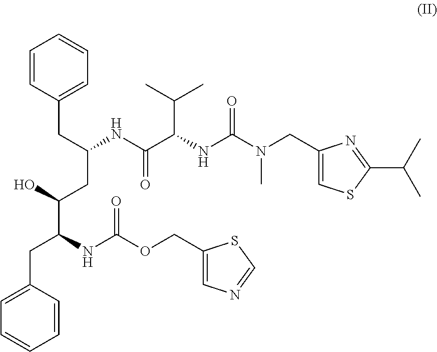 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

Unexpectedly, it has now been found that compounds of Formula (I), (II), and (III) for combined dosing may provide alternative/improved HCV therapy.

### DESCRIPTION OF THE INVENTION

The present invention relates to a combination comprising (i) the compound of formula I:

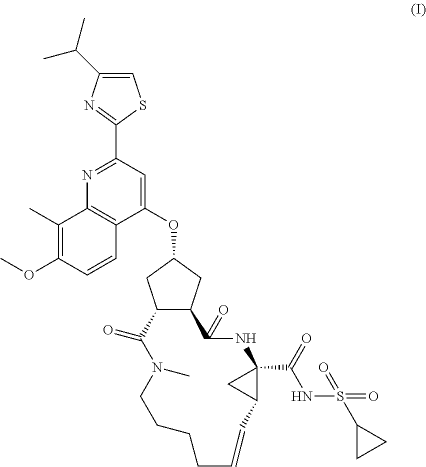 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]

or a pharmaceutically acceptable salt thereof,

(ii) the compound of formula II:

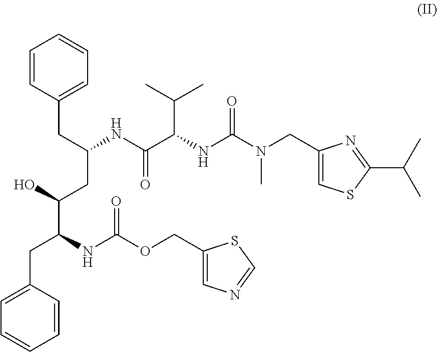 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

or a pharmaceutically acceptable salt thereof,

and (iii) and the compound of formula III:

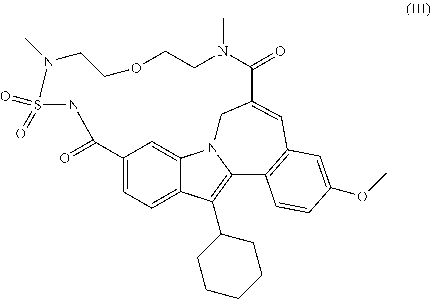 [SMILES: COc1ccc2c(c1)C=C1Cn3c-2c(C2CCCCC2)c2ccc(cc23)C(=O)NS(=O)(=O)N(C)CCOCCN(C)C1=O]

or a pharmaceutically acceptable salt thereof, wherein the amount of the compound of formula I is about 25 to 100 mg, the amount of the compound of II is about 10 to 50 mg and the amount of the compound of formula III is about 100 to 750 mg.

More generally, the present invention relates to a combination of a macrocyclic NS3/4A protease inhibitor of HCV, a HCV NS5B polymerase inhibiting non-nucleoside and ritonavir.

The invention further relates to a combination of compounds of Formula (I), (II), and (III) further comprising another HCV antiviral selected from an HCV polymerase inhibitor, an HCV protease inhibitor, an inhibitor of another target in the HCV life cycle, and immunomodulatory agent, an antiviral agent, and combinations thereof.

In addition, the invention relates to a product comprising the compound of formula I, II and the compound of formula III, as a combined preparation for simultaneous, separate or sequential use in HCV therapy. In one embodiment, such combination product essentially consists of compound of formula I, II and the compound of formula III. In another embodiment, such combination product consists of compound of formula I, II and the compound of formula III.

Furthermore, the invention relates to a pharmaceutical composition comprising a combination of compounds of Formula (I), (II), and (III), and a pharmaceutically acceptable carrier.

Additionally, the invention relates to a method for treating HCV infection comprising administering to a patient in need of such treatment a combination or a pharmaceutical composition according to the invention wherein the compound of formula I, II and the compound of formula III are in separate dosage forms, or in a single dosage form.

Finally, the invention relates to use of a combination pharmaceutical composition according to the invention in the prevention and treatment of HCV infection or diseases associated therewith.

Whenever used hereinafter, the term “compounds of formula (I), (II) or (III)”, or “the present compounds” or similar term is meant to include the compounds of general formula (I), (II) or (III) and pharmaceutically acceptable salt forms thereof.

The compounds of formula I, formula II or formula III may be used in pharmaceutically acceptable salt forms or in free (i.e. non-salt) form. Salt forms can be obtained by treating the free form with an acid or base. Of interest are the pharmaceutically acceptable acid and base addition salts, which are meant to comprise the therapeutically active non-toxic acid and base addition salt forms that the compounds are able to form. The pharmaceutically acceptable acid addition salts of the compounds of formula I, formula II or formula III can conveniently be obtained by treating the free form with such appropriate acid. Appropriate acids comprise, for example, inorganic acids such as hydrohalic acids, such as hydrobromic acid, or in particular hydrochloric acid; or sulfuric, nitric, phosphoric and the like acids; or organic acids such as, for example, acetic, propanoic, hydroxyacetic, lactic, pyruvic, oxalic, malonic, succinic, maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. The compounds of formula I may also be converted into the pharmaceutically acceptable metal or amine addition salt forms by treatment with appropriate organic or inorganic bases. Appropriate base salt forms comprise, for example, the ammonium salts, the alkali and earth alkaline metal salts, e.g. the lithium, sodium or potassium salts; or the magnesium or calcium salts; salts with organic bases, e.g. the benzathine, N-methyl-D-glucamine, hydrabamine salts, and salts with amino acids such as, for example, arginine, lysine, and the like. The term addition salt form is meant to also comprise any solvates that the compounds of formula I, formula II or formula III, as well as the salts thereof, may form. Such solvates are, for example, hydrates, alcoholates, e.g. ethanolates, and the like.

The amounts of the compound of formula I in the combinations of the invention that are administered on a daily basis may vary from about 1 mg to about 2500 mg, about 5 mg to about 1000 mg, or from about 10 mg to about 500 mg, or from about 25 mg to about 250 mg, or from about 25 mg to about 200 mg. Examples of daily amounts of the compound of formula I are 25 mg, 50 mg, 75 mg, 100 mg, 125 mg, 150 mg, 200 mg, and 400 mg.

The amounts of the compound of formula II that are administered on a daily basis may vary from about 10 mg to about 50 mg, or from about 15 mg to about 40 mg, or from about 15 mg to about 30 mg, or from about 20 mg to about 20 mg. Examples of daily amounts of the compound of formula II are 15 mg, 18 mg, 20 mg, 22 mg, 24 mg 28 mg and 30 mg.

The amounts of the compound of formula III that are administered on a daily basis may vary from about 10 mg to about 2000 mg, or from about 20 mg to about 1000 mg, or from about 50 mg to about 750 mg, or from about 300 mg to about 600 mg, or from about 125 mg to about 500 mg. Examples of daily amounts of the compound of formula III are 100 mg, 150 mg, 200 mg, 250 mg, 300mg, 350 mg, 400 mg, 450 mg 500 mg, 600 mg, 750 mg and 1000 mg.

In an embodiment of the invention (for instance, a particularly preferred embodiment), there is provided a combination comprising (or consisting of) a compound of formula I in an amount from between about 50 mg and 100 mg (preferably 75 mg), a compound of formula II in an amount from between about 20 mg and about 40 mg (preferably 30 mg) and a compound of formula III in an amount from between about 300 mg to about 600 mg (preferably between about 400 and 500 mg, e.g. 450 mg). In this respect, the combination preferably comprises 75 mg of a compound of formula I, 30 mg of a compound of formula II and 450 mg of a compound of formula III. Such amounts/doses refer to the daily dose, and where the compounds I, II and III are separate doses forms, the doses may be taken simultaneously or sequentially (preferably, if they are separate dosage forms the doses are taken within 1 hour of each other, e.g. within 30 minutes of each other).

All amounts mentioned in this and the following paragraphs refer to the free form (i.e. non-salt form). The above values represent free-form equivalents, i.e. quantities as if the free form would be administered. If salts are administered the amounts need to be calculated in function of the molecular weight ratio between the salt and the free form.

The above mentioned daily doses are calculated for an average body weight of about 70 kg and should be recalculated in case of paediatric applications, or when used with patients with a substantially diverting body weight.

The dosages may be presented as one, two, three or four or more sub-doses administered at appropriate intervals throughout the day. The dosage used preferably corresponds to the daily amount of the compound of formula I, formula II or formula III mentioned above, or a sub-dose thereof, such as ½, ⅓, or ¼ thereof. A dosage form may contain the compound I, the compound II, or the compound III, or all three together, in an amount equal to the ranges or quantities mentioned in the previous paragraphs, either in separate formulations or in a combined formulation. Such combined formulation is preferred.

In the instance where all three compounds, of formula I formula II and formula III are to be administered once daily, this can be accomplished by administering three separate doses, one with compound I, the other with compound II, and the third with compound III, or by administering a combined dose containing compound I and compound II and compound III.

Administration of dosages may be by separate dosage forms, i.e. dosage forms only containing compound I or only compound II or only compound III; or by combined dosage forms containing active ingredients I, II and III. Also, a mix of using a combined dosage form and separate dosage forms can be used. Dosage forms that can be administered are described hereinafter, oral dosage forms, in particular tablets or capsules being preferred.

Active ingredients may be formulated in pharmaceutical compositions either separately or as a combined pharmaceutical composition. In the latter instance, there is provided a pharmaceutical composition comprising a therapeutically effective amount of the compound of formula I, or a pharmaceutically acceptable salt thereof, and the compound of formula II, or a pharmaceutically acceptable salt thereof, and the compound of formula III, or a pharmaceutically acceptable salt thereof, the foregoing being as specified herein, and a pharmaceutically acceptable carrier. A therapeutically effective amount in this context is an amount sufficient to act in a prophylactic way against, or to stabilize or to reduce HCV infection, in infected subjects or subjects being at risk of being infected. Therapeutically effective amounts may in particular correspond to the amounts mentioned above for administration on a daily base or of the subdoses thereof in ease of multiple daily administrations.

In a further aspect, this invention relates to a process of preparing a pharmaceutical composition as specified herein, which comprises intimately mixing a pharmaceutically acceptable carrier with a therapeutically effective amount of the compound of formula I, or a pharmaceutically acceptable salt thereof, and an effective amount of the compound of formula II, or a pharmaceutically acceptable salt thereof, and a therapeutically effective amount of the compound of formula III, or a pharmaceutically acceptable salt thereof.

The combinations provided herein may also be formulated as a combined preparation for simultaneous, separate or sequential use in HCV therapy. In such a case, the compound of formula I is formulated in a pharmaceutical composition containing other pharmaceutically acceptable excipients, and the compound of formula II is formulated separately in a pharmaceutical composition containing other pharmaceutically acceptable excipients, and the compound of formula III is formulated separately in a pharmaceutical composition containing other pharmaceutically acceptable excipients. Conveniently, these separate pharmaceutical compositions can be part of a kit for simultaneous, separate or sequential use.

The individual components of the combination of the present invention can be administered simultaneously or separately at different times during the course of therapy or concurrently in divided or single combination forms.

Therefore, the compounds of formula I, II and III, individually or combined, may be formulated into various pharmaceutical compositions suitable for administration purposes. In these, a therapeutically effective amount of the particular compound, or of all three compounds, is combined with a pharmaceutically acceptable carrier, which carrier may take a wide variety of forms depending on the form of preparation desired for administration. Pharmaceutical compositions may be prepared as medicaments to be administered orally, parenterally (including subcutaneously, intramuscularly, and intravenously), rectally, transdermally, bucally, or nasally. Suitable compositions for oral administration include powders, granulates, aggregates, tablets, compressed or coated pills, dragees, sachets, hard or gelatin capsules, syrups and suspensions. Suitable compositions for parenteral administration include aqueous or non-aqueous solutions or emulsions, while for rectal administration suitable compositions for administration include suppositories with a hydrophilic or hydrophobic vehicle. For topical administration there can be used suitable transdermal delivery systems and for nasal delivery there can be used suitable aerosol delivery systems.

For example, in preparing the compositions for oral administration, any of the usual pharmaceutical media may be employed such as, for example, water, glycols, oils, alcohols and the like in the case of oral liquid compositions such as suspensions, syrups, elixirs, emulsions and solutions; or solid carriers such as starches, sugars, kaolin, lubricants, binders, disintegrating agents and the like in the case of solid compositions. For parenteral compositions, the carrier will usually comprise sterile water, at least in large part, though other ingredients, such as solubilizers, emulsifiers or further auxiliaries may be added thereto. Injectable solutions may be prepared in which the carrier comprises saline solution, glucose solution or a mixture of both. Injectable suspensions may also be prepared in which case appropriate liquid carriers, suspending agents and the like may be employed. Also included are solid form preparations intended to be converted, shortly before use, to liquid form preparations such as powders for reconstitution. In the compositions suitable for percutaneous administration, the carrier optionally comprises a skin penetration enhancing agent and/or a wetting agent, optionally combined with suitable skin-compatible additives in minor proportions. The compounds of the compounds of formula I, II and III, or combinations thereof, may also be administered via oral inhalation or insufflation by formulations suited for this type of administration such as a solution, a suspension or a dry powder. Suitable pharmaceutical compositions for administration in the form of aerosols or sprays are, for example, suspensions of the compound of the compounds of formula I, II and III, or both, in a pharmaceutically acceptable liquid carrier, such as ethanol or water, or a mixture thereof. If required, the formulation can also additionally contain other pharmaceutical auxiliaries such as surfactants, emulsifiers and stabilizers as well as a propellant. Such a preparation customarily contains the active compound in a concentration from approximately 0.1 to 50% by weight.

The pharmaceutical compositions may contain the active ingredient of formula I, or of formula II, or of formula III, or all three combined.

The pharmaceutical compositions may be conveniently presented in unit dosage form for ease of administration and uniformity of dosage. Examples include tablets (including scored or coated tablets), capsules, pills, suppositories, powder packets, wafers, injectable solutions or suspensions and the like, and segregated multiples thereof. Of interest are solid dosage forms for oral administration such as tablets on capsules.

The solid dosage forms in unit dose form may be packed in any known package, blister packs being preferred, in particular for tablets and capsules. Where the compound of formula I, of formula II and of formula III are formulated separately, they could be packed in separate blisters, but one blister could as well comprise unit dose forms of the compound I as of the compound II as of the compound III, for example one row with units of compound I and another with compound II, and another with compound III. Other possibilities may be possible as well.

The combinations of this invention may be used to treat HCV infections as well as diseases associated with HCV. The diseases associated with HCV include progressive liver fibrosis, inflammation and necrosis leading to cirrhosis, end-stage liver disease, and HCC (hepatocellular carcinoma).

The in vitro antiviral activity against HCV of the compound of formula I or of formula III can be tested in a cellular HCV replicon system based on Lohmann et al. (1999) Science 285:110-113, with the further modifications described by Krieger et al. (2001) Journal of Virology 75: 4614-4624 (incorporated herein by reference), which is further exemplified in the examples section. This model, while not a complete infection model for HCV, is widely accepted as the most robust and efficient model of autonomous HCV RNA replication currently available. The in vitro antiviral activity against HCV can also be tested by enzymatic tests.

The combination of the compound of formula I, formula II and the compound of formula III, as specified herein, is useful in the treatment of warm-blooded animals, in particular humans, infected with HCV, and for the prophylaxis of HCV infections.

The present invention therefore furthermore relates to a method of treating a warm-blooded animal, in particular a human, infected by HCV, or being at risk of infection by HCV, said method comprising the administration of an anti-HCV effective amount of a combination of the compound of formula I, of formula II and the compound of formula III, as specified herein. The present invention provides as well a method of treating HCV-related conditions or preventing HCV-related conditions in a mammal comprising administering an anti-virally effective amount of a combination of the compound of formula I, of formula II and the compound of formula II, of formula III, as specified herein.

The pharmacokinetics of the compound combination according to the invention may be described using pharmacokinetic parameters known to the person skilled in the art.

Examples of such parameters include: t_{1/2} (half life), Cₘᵢₙ (minimal concentration, trough concentration), Cₘₐₓ (maximal concentration), AUC (area under the curve), Tₘₐₓ (time to maximal concentration), (Cₛₛ) steady state concentration.

The combinations of the present invention may be used as medicaments. The present invention also relates to the use of a combination, as described herein, for the manufacture of a medicament for the treatment or the prevention of HCV infection or HCV related conditions.

In a further aspect, the invention relates to a product containing the compound of formula I, formula II and the compound of formula III, and optionally another anti-HCV compound, as a combined preparation for simultaneous, separate or sequential use in the treatment of HCV infections.

The combinations of the present invention in turn may be combined with one or more further anti-HCV compounds. Of interest are combinations with IFN-α (pegylated or not) and/or ribavirin or HCV nucleotide polymerase inhibitors.

The other agents that may be co-administered with the combinations of the present invention may be administered as separate formulations or may be co-formulated with one or more of the active ingredients of formula I, of formula II or of formula III.

As used herein, the term “about” has its conventional meaning. In particular embodiments, when in relation to a numerical value, it may be interpreted to mean the numerical value ±10%, or ±5%, or ±2%, or ±1%, or ±0.5%, or ±0.1%. In other embodiments, the precise value is meant, i.e. by leaving out the word “about”.

### EXAMPLES

The following examples are intended to illustrate the present invention and not to limit it thereto.

The pharmacokinetic variables for the compound of formula (I) and (III) administered alone where compared when they were co-administered in a combination of (I), (II) and (III).

All tables indicate Geometric mean (CV %) pharmacokinetic parameters and might be the result of multiple studies. The compounds are indicated by their respective numbers (I, II or III) in the tables below.

The exposure of ritonavir (compound of Formula II, RTV) by the combined administration of compounds of Formula (I) and (III) is indicated in the table 1 below and is an exposure generated in healthy subjects at steady state (between brackets the Coefficient of Variation is given).

TABLE 1

| | Cₘₐₓ | Cₘᵢₙ | AUC_{24 h} |
|---|---|---|---|
| Dose (qd) | ng/ml | ng/ml | ng · h/ml |
| 20 mg RTV + 50 mg (I) + | 75 (69%) | <LOQ | 506 (79%) |
| 300 mg (III) | | | |

A comparison is made between an administration of compound of Formula (III) 1000 mg alone, versus 300 mg compound of formula (III) in combination with 50 mg of compound of Formula (I) and 20 mg of Ritonavir (compound of Formula (II). The results are indicated in table 2 below.

TABLE 2

| | Cₘₐₓ | Cₘᵢₙ | AUC_{24 h} |
|---|---|---|---|
| Dose | μg/ml | μg/ml | μg · h/ml |
| 1000 mg bid (III) | 5.22 (27%) | 0.16 (42%) | 40.1 (33%) |
| 300 mg (III) + | 6.57 (60%) | 0.21 (86%) | 52.2 (56%) |
| 50 mg (I) + 20 mg (II) qd | | | |

A comparison is made between administration of compound of Formula (I) 150 mg alone versus 300 mg compound of formula (III) in combination with 50 mg of compound of Formula (I) and 20 mg of Ritonavir (compound of Formula (II) according to the following protocol.
150 mg qd (I) given for 14 days to 10 healthy subjects
Washout of 14 days
50 mg qd (I), 300 mg qd (III), 20 mg qd ritonavir (II) given for 14 days to the same 10 healthy volunteers.

The results are shown in table 3 below.

TABLE 3

| | Cₘₐₓ | Cₘᵢₙ | AUC_{24 h} |
|---|---|---|---|
| Dose (qd) | ng/ml | ng/ml | ng · h/ml |
| 150 mg (I) | 4746 | 1405 | 71241 |
| | (64%) | (104%) | (78%) |
| 50 mg (I) | 1458 | 375 | 19969 |
| 300 mg (III) 20 mg RTV | (83%) | 51%) | (66%) |

It is apparent from the tables above that specific combinations of dosages of compounds of Formula (I), (II) and (III) are required to manage effects that the compounds have on each other with regard to in vivo exposure. Also, a low dose of ritonavir (lower than 50 mg, for example 20 mg), provides for efficient boosting of the specific combination of compounds of Formula (I) and (III).

The combination may have the advantage that it may be more efficacious than, improve compliance due to less frequent administration, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

1. A combination comprising (i) the compound of formula I: 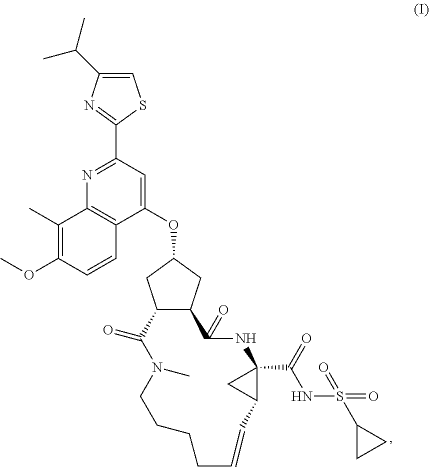 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]
or a pharmaceutically acceptable salt thereof,
(ii) the compound of formula II: 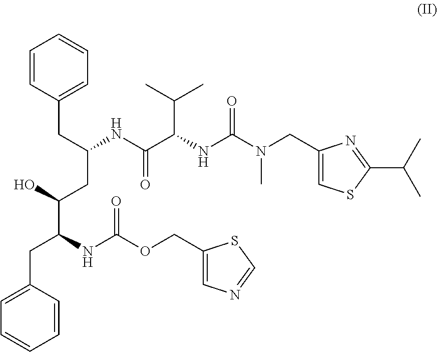 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof,
and (iii) and the compound of formula III: 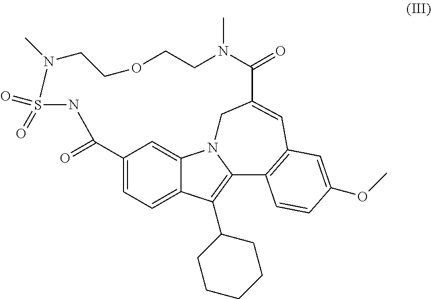 [SMILES: COc1ccc2c(c1)C=C1Cn3c-2c(C2CCCCC2)c2ccc(cc23)C(=O)NS(=O)(=O)N(C)CCOCCN(C)C1=O]
or a pharmaceutically acceptable salt thereof, wherein the amount of the compound of formula I is about 25 to 100 mg, the amount of the compound of II is about 10 to 50 mg and the amount of the compound of formula III is about 100 to 750 mg.

2. The combination according to claim 1 wherein the amount of the compound of formula II is about 15 to 30 mg.

3. The combination according to claim 1, wherein the amount of the compound of formula I is about 40 to 80 mg.

4. The combination according to claim 1, wherein the amount of the compound of formula III is about 200-600 mg.

5. The combination according to claim 1, wherein the amount of the compound of formula I is about 75 mg, the amount of the compound of II is about 25 mg and the amount of the compound of formula III is about 300 mg.

6. The combination according to claim 1, further comprising another HCV antiviral selected from an HCV polymerase inhibitor, an HCV protease inhibitor, an inhibitor of another target in the HCV life cycle, an immunomodulatory agent, an antiviral agent, and combinations thereof.

7. The combination of claim 1, as a combined preparation for simultaneous, separate or sequential use in HCV therapy.

8. A pharmaceutical composition comprising a combination as claimed in claim 1, and a pharmaceutically acceptable carrier.

9. A method for treating HCV infection comprising administering to a patient in need of such treatment a combination according to claim 1, wherein the compound of formula I, II and the compound of formula III are in separate dosage forms, or in a single dosage form.

10. The method according to claim 9 wherein the separate dosage forms are administered about simultaneously.

11. (canceled)
